(19) United States
(12) Patent Application Publication
(10) Pub. No.: US 2024/0139364 A1
(43) Pub. Date: May 2, 2024

(54) AUTONOMOUS ROBOTIC DISINFECTION SYSTEM

(71) Applicant: AVALON STERITECH LIMITED, Hong Kong (CN)

(72) Inventors: Wai Hong HO, Hong Kong (CN); Johnson Yiu-Nam LAU, Houston, TX (US); Kar Yun LEE, Hong Kong (CN)

(73) Assignee: AVALON STERITECH LIMITED, Hong Kong (CN)

(21) Appl. No.: 18/280,457

(22) PCT Filed: Mar. 2, 2022
(86) PCT No.: PCT/IB2022/051835

§ 371 Date: Sep. 5, 2023

Related U.S. Application Data
(60) Provisional application No. 63/156,243, filed on Mar. 3, 2021.

(51) Int. Cl. A61L 2/24; A61L 2/22

(52) U.S. Cl. CPC A61L 2/24; A61L 2/22; A61L 2202/14; A61L 2202/16; A61L 2202/25

(57) ABSTRACT

A cleaning and disinfection system. More particularly, aspects of the invention relate to a system and method for enhancing the Environment, Social, Governance of a company through autonomous cleaning and disinfection mobile robots. In particular, cleaning, disinfecting and sterilizing public space in mass transportation, for example railway system, by using the autonomous cleaning and disinfection mobile robot.

### FIELD OF INVENTION

This invention generally relates to a cleaning and disinfection system. More particularly, aspects of the invention relate to a system and method for enhancing the Environment, Social, Governance of a company through autonomous cleaning and disinfection mobile robots. In particular, cleaning, disinfecting and sterilizing public space in mass transportation, for example railway system, by using the autonomous cleaning and disinfection mobile robot.

### BACKGROUND

Millions of people are using public transits every day. Therefore, it is essential to keeping the public space of the public transits clean. The public transits service providers usually perform one major cleaning at once a while and minor cleaning a couple of times per day. Cleanings are usually carried out manually and the cleaning equipment used mainly specialized in removing visible foreign matter (e.g. dust and trash) only. Vacuum cleaner, mop and sweeper often are the most common cleaning equipment used.

After the beginning of the COVID-19 pandemic, the public confidence in public transportation drops significantly due to hygiene concerns. The government also concerns public transits may increase the spreading of COVID-19 or other virus in the community. In view of that, the public transits service providers begin to disinfect the public area to reduce the risk of spreading disease in public transits.

Cleaning is different from disinfection. Cleaning removes visible foreign matters, including dirt, dust, and crumbs from surfaces or object. Disinfection uses disinfectants to kill germs on surface and object. Disinfection does not necessarily clean dirty surfaces.

Like the cleaning operations, disinfection operations in the public transits are carried out manually. Manual disinfection has the following disadvantages:

Inconsistency and unreliability. It is difficult to ensure an cleaning operator cleans and disinfects every spot in a public space consistently. Overuse of disinfectant during the cleaning and disinfection happens all the time as the cleaning operators has no idea how much they use at certain spot during disinfection. Overuse will create unnecessary associated risks to the public.

Increase in risk of infection. A cleaning operator is at risk to be invaded by pathogenic agent even he/she fully wears protective gears when he/she disinfect an area. As a result of that, the risk of infection in the community also increases due to the interaction among the cleaning operator and the community.

Increase of amount of waste. Every cleaning operator has to wear disposable protective gears every time they disinfect an area. Therefore, the amount of waste increases as the number of disinfection performed increases.

Time, cost and employees' injuries. Regular cleaning and disinfection are needed to ensure cleanliness in an area. However, there are many areas which are difficult to be reached by human, for example, HVAC conduits. It takes a lot of human power as well as time to clean and/or disinfecting it. The increase in time and cost may make frequent cleaning in those areas impracticable. Further, employees may get injured when cleaning and disinfecting those area.

The above mentioned disadvantages fail to improve Environment, Social, and Governance (ESG) ratings of a company as they fail to reduce pollution and waste. It also exposes the cleaning operator to any potential infection. Companies always look for way to improve their ESG ratings. This is because the companies that score well on ESG ratings tend to better anticipate future risks and opportunities. Those companies also tend to deliver long-term value and long-term strategic thinking.

### SUMMARY

In light of the foregoing background, it is an aspect of the present invention to provide an autonomous cleaning and disinfection mobile robotic system to tackle all the disadvantages mentioned in the background.

In particular, it is an aspect of the present invention to enhance consistency and reliability when it comes to cleaning and disinfection. The robot of the present invention optimize the disinfection by precisely controlling the volume of disinfectant to be applied to the surrounding based on the condition, for example, temperature, humidity atmospheric pressure, air circulation and the distance between the ceiling of the public space and the disinfection nozzle of the robot. It helps striking the perfect balance between biocidal efficacy and over-use.

It is yet another aspect of the present invention to reduce the risk of infection and the amount of protective gear (e.g., waste). The robot of the present invention performs the cleaning and disinfection itself. The cleaning operator operates remotely from the contaminated area. The robot may avoid obstacles while following a predetermined path for cleaning and disinfection. The robot does not require any protective gear to perform disinfection and may also perform self-cleaning protocol to disinfect itself immediately after the disinfection to protect the cleaning operator from contamination. Therefore, the risk of infection and the amount of protective gear (e.g., waste) are significantly reduced.

It is yet another aspect of the present invention to reduce the time and cost of cleaning and disinfection, helping to bring back public confidence in public transits. Multiple robots may be deployed to clean and disinfect an area simultaneously. The multiple robots may communicate with each other to clean an area to optimize the cleaning and disinfecting efficiency. The robots may also be deployed at places where it is hard to be reached by human, for example heating, ventilation and air conditioning (HVAC) conduits. Since the difficulties for clean such area is reduced due to minimal human involvement, it may be cleaned more often and the risk of injury may be reduced. Frequent cleaning and disinfecting the area in connection with the public transits is an important element to re-establish public confidence in public transits.

It is yet another aspect of the present invention to improve ESG ratings of a company through reduction of waste, pollution and injury and increase of customer satisfaction and employees' working environment.

Additional aspects of the invention will be set forth in part in the description which follows and, in part, will be obvious from the description, or may be learned by practice of the invention.

Accordingly, embodiments of the present invention, in one aspect, may be an automated cleaning and disinfection robot comprising an automated navigation system capable of navigating through an enclosed area and reaching predetermined spots thereof while avoiding obstacles; a fluid storage for storing Quaternary Ammonium Compounds (QACs) and/or other disinfectant (“Disinfectant”), wherein an adherence additives may be added to such Disinfectant to enhance the ability for allowing Disinfectant to adhere to a surface thus providing extra and prolonged protection against the pathogen agents; a plurality of sensors to (i) detect obstacles; (ii) identify contaminated spot; and (iii) measure the content of biocidal in the surrounding area to ensure striking the perfect balance between biocidal efficacy and over-use; a flexible propulsion system that can move the robot through different space within a public transits; a disinfection module capable of removing contaminates and pathogenic agents on a predetermined surface; and a non-transitory computer readable storage medium configured to store instructions that when executed is configured to cause a processor to execute the instructions for (i) obtaining at least one environment parameter including at least the temperature, the humidity and the pressure, and (ii) optimizing biocidal efficacy and the amount of Disinfectant used based on the environment parameter, wherein the robot is configured to (i) operate in an environmentally friendly approach; and (ii) prevent contamination/cross infection due to the prolonged adherence of the Disinfection chemical on a surface.

In another aspect, the automated cleaning and disinfection robot further comprises an atomization system energized by pressurized air, wherein the spraying pressure helps create Disinfectant droplets; an electrostatic sprayer having an nozzle with an opening size selected from 10, 20, 40 or 80 nm for controlling the droplet size sprayed into the air as well as onto a surface; a pressure control unit to control the spraying pressure thereby controlling the distance and/or height traveled by the droplets; and an optional mechanical arm configured to extend the electrostatic sprayer away from the robot, wherein the combination of the nozzle and the pressure control unit helps reducing the amount of Disinfectant used, and wherein the concentration of the Disinfectant may be adjusted based on the specific application.

### BRIEF DESCRIPTION OF DRAWINGS

Persons of ordinary skill in the art may appreciate that elements in the figures are illustrated for simplicity and clarity so not all connections and options have been shown. For example, common but well-understood elements that are useful or necessary in a commercially feasible embodiment may often not be depicted in order to facilitate a less obstructed view of these various embodiments of the present disclosure. It may be further appreciated that certain actions and/or steps may be described or depicted in a particular order of occurrence while those skilled in the art may understand that such specificity with respect to sequence is not actually required. It may also be understood that the terms and expressions used herein may be defined with respect to their corresponding respective areas of inquiry and study except where specific meanings have otherwise been set forth herein.

FIG. 1 is a schematic view of autonomous cleaning and disinfection mobile robots according to one embodiment.

FIG. 2 is a schematic view of autonomous cleaning and disinfection mobile robots for public transits according to one embodiment.

FIG. 3 is a schematic view of autonomous cleaning and disinfection mobile robots for HVAC conduits according to one embodiment.

FIG. 4 is a schematic view of autonomous cleaning and disinfection mobile robots for scrubbing floor according to one embodiment.

### DETAILED DESCRIPTION

Embodiments may now be described more fully with reference to the accompanying drawings, which form a part hereof, and which show, by way of illustration, specific exemplary embodiments which may be practiced. These illustrations and exemplary embodiments may be presented with the understanding that the present disclosure is an exemplification of the principles of one or more embodiments and may not be intended to limit any one of the embodiments illustrated. Embodiments may be embodied in many different forms and should not be construed as limited to the embodiments set forth herein; rather, these embodiments are provided so that this disclosure may be thorough and complete, and may fully convey the scope of embodiments to those skilled in the art. Among other things, the present invention may be embodied as methods, systems, computer readable media, apparatuses, or devices. Accordingly, the present invention may take the form of an entirely hardware embodiment, an entirely software embodiment, or an embodiment combining software and hardware aspects. The following detailed description may, therefore, not to be taken in a limiting sense.

### First Embodiment

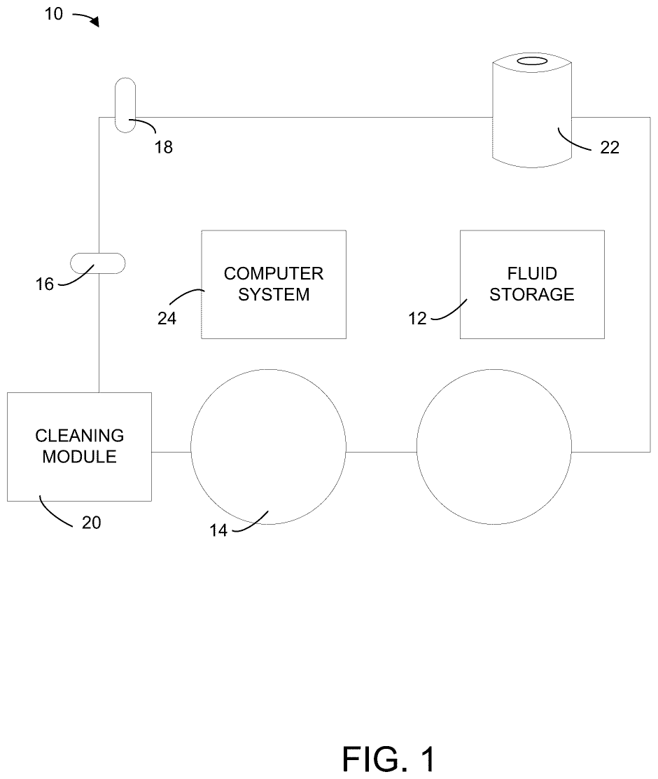

Referring to FIG. 1, the first embodiment of an automated cleaning and disinfection robot 10 comprises at least one fluid storage 12 configured to hold Quaternary Ammonium Compounds (QACs) and/or any other suitable disinfectant (“Disinfectant”), at least one wheel 14, at least one obstacle sensor 16 installed at the front or the side of the robot 10 configured to detect any obstacle in its path as it travels, at least one disinfectant sensor 18 configured to obtain the concentration of dispensed Disinfectant in an area and/or volume, a cleaning module 20 configured to remove visible foreign matters from a surface, a disinfection module 22 configured to apply Disinfectant to kill airborne germs and/or germs on the surface and a computer system 24 configured to receive the signals from the sensors 16, 18, control the disinfection module 22 based on the signal received from the disinfectant sensor 18, and navigate through a predetermined area while avoiding obstacle based on the signal from the obstacle sensor 16.

There are many factors affecting the biocidal efficacy, for example, the materials to be disinfected, the temperature, the humidity and the pressure. The computer system 24 of the present invention may use machine learning to optimize the biocidal efficacy and the amount of Disinfectant used based on the surrounding factors thereby avoiding over-use of Disinfectant. Optimization of Disinfectant can reduce the pollution and the associated risk to the public.

In some embodiments, an adherence chemical (e.g. polymer) may be added into the Disinfectant to allow it to stay on the surface longer.

In some embodiments, the robot 10 may include any rechargeable battery. In yet some embodiment, the robot 10 includes lithium battery.

In some embodiments, the robot 10 may include motor configured to drive the wheel 14.

In some embodiments, the obstacle sensor 16 may be a medium range scanner and may give the robot 10 a 360 degree visibility for at least 5 meters. In some embodiments, the scanner may be light sourced (e.g. laser) or an audio-based (e.g. sonar). In some embodiments, the obstacle sensor 16 may be a camera and/or sonar transducer.

### Second Embodiment

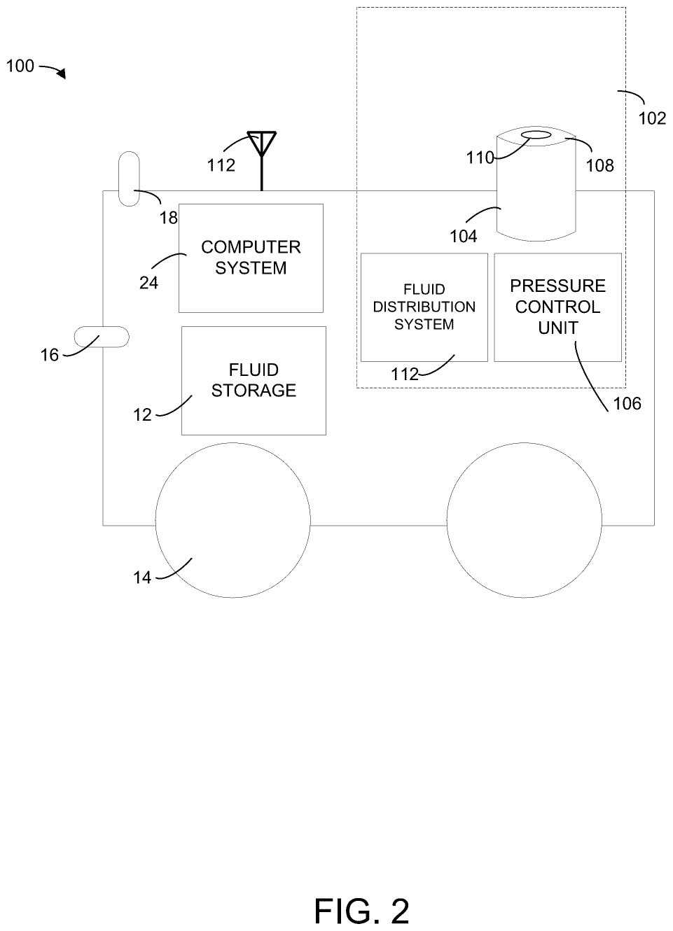

Now refer to the automatic cleaning and disinfection robot 100 according to a second embodiment of the present invention. The robot 100 in this embodiment is specific for cleaning and disinfecting enclosed space in connection with public transits. In some specific embodiments, this robot is for cleaning and disinfecting enclosed space in connection with railway systems. As shown in FIG. 2, the robot 100 of this specific embodiment comprises at least one fluid storage 12 configured to hold Disinfectant, at least one obstacle sensor 16 installed at the front of the robot configured to detect any obstacle in its path as it travels, at least one disinfectant sensor 18 configured to measure the concentration of dispensed Disinfectant in an area and/or an enclosed area, a disinfection module 102 configured to spray the Disinfectant into the air and surface around the robot 200 and a computer system 24 configured to receive the signals from the sensors, control the disinfection module 102 based on the signal from the disinfectant sensor 18, and navigate through a predetermined area while avoiding obstacle based on the signal from the obstacle sensor 16. The disinfection module 102 comprises a fluid atomization system 104 energized by pressurized air, a pressure control unit 106 configured to control the spraying pressure of the fluid atomization system 104, an electrostatic sprayer 108 having a nozzle 110, a distribution system 112 connected to the fluid storage 12 configured to feed the fluid in the fluid storage 12 to the fluid atomization system 104 and the electrostatic sprayer 108. The size of the nozzle 110 helps to regulate the droplet size of the Disinfectant sprayed into the air and/or the surface of its surrounding. The pressure control unit 106 further controls the spraying pressure to control the distance and/or height traveled by the Disinfection droplets. In some embodiments, the spraying is ranged from 0.1 to 30 meters for Disinfection having a concentration up to 600 ppm. The spraying range will drop for Disinfection having a concentration higher than 600 ppm. By adjusting the size of the nozzle 110 and the spraying pressure, the biocidal efficacy and the amount of Disinfectant used may be optimized. Thereby the amount of Disinfectant used may be reduced.

The computer system 24 may control the robot to complete cleaning and disinfection on its own after at least one parameter is given, which might include but not limited to cleaning and disinfection instruction plan and/or the parameters of the enclosed area to be cleaned and disinfected (including but not limited to the size and shape of the area, the height of the ceiling, ventilation ports, the materials of the floor and the wall, the position of the doors and seats, temperature, pressure and humidity). The computer system 24 may then create a cleaning and disinfection plan based on the plan and the parameters to optimize the biocidal efficacy and the use of Disinfectant.

In another embodiment, aspects of the invention may provide the government entities or clients a direct channel or platform to provide instructions or orders to the robot 100. For example, to improve ESG ratings, aspects of the invention may enable its computer system 24 to receive instructions from the clients. For example, the client may be a public transportation provider, such as a subway or bus operator. As these clients usually may be a branch or a contractor of a government, aspects of the invention may intelligently convert information quickly from these clients to the computer system 24 to control the robot 100.

In one example, suppose the public transportation provider (e.g., MTR in Hong Kong) may work with a number of government entities to implement government rules, regulations, policies, etc. In another embodiment, the provider may also need to accommodate to emergency situations such as unexpected crowds, health hazard situations, pandemic, protests, demonstrations, etc. As such, the provider may publish or generate one of the following:

Public notices, Bulletins, Circulars, etc.

With these published information, the computer system 24 may receive the information automatically via push and may identify the necessary format or style of the published information. In other words, the computer system 24 may consume the information and extract the necessary information that may be converted to the computer-implemented instructions that may be executed by the computer system 24 so that the robot 100 may be operational. It is to be understood that the published information may include human readable content and not machine-readable content. For example, the published information may be press releases or other government-published information that has a given format.

In another embodiment, the published information may be color coded or may include quick response (QR) code that may include that may be interpreted by the computer system 24 as instructions for the robot 100.

In yet another embodiment, aspects of the invention may incorporate machine learning or artificial intelligence that may further fine-tune the interpretation of the information. In yet a further embodiment, as the information is received by the computer system 24, another copy of the information is sent to a service provider administration so that the administration may perform manual edits or corrections to the interpretation by the computer system 24. For example, the administrator or contractor who handles the information may receive a copy and then may assign programmers or technicians who hand-code the instructions for the robot 100 to execute.

In yet another embodiment, aspects of the invention may further include application programming interface (API) that may receive the information from the clients more directly.

With such implementation, the client may precisely and timely provide instructions for the robot 100 to execute that meet the requirements from the government. This approach further may increase or improve ESG ratings. For example, the requirements defined by government contracts may be provided to the computer system 24 through the interpretation process and the interpretation may extract, parse or identify the information suitable for the computer system 24.

In one aspect, the computer system 24, after interpretation, may enable the deployment of the robot 100. In one example, the deployment may be delayed or scheduled. In yet another example, the deployment may be done immediately or substantially simultaneously. In another example, the deployment may be programmed as aided by the administrator of the contractor.

A computer-based model relating the parameters of the enclosed area to be cleaned and the cleaning parameters is stored in the computer system 24. The computer system 24 may use machine learning to optimize the biocidal efficacy and the amount of Disinfectant used based on local data. The biocidal efficacy and the use of Disinfectant may be optimized through controlling the cleaning parameters which includes but not limited to, controlling the amount of disinfectant use, droplet size, the spraying pressure (thus controlling the Disinfectant droplet travel distance), the disinfection time and the angle of which the disinfectant is applied.

In some embodiment, the robot may include additional sensor to measure the temperature, pressure and related humidity. The computer system 24 may use machine learning to optimize the biocidal efficacy and the amount of Disinfectant used based on real time data measured by the thermometer, barometer and/or the humidity meter.

The computer system 24 may further use machine learning to identify the materials and/or type of objects (for example, the floor, the wall, the doors, seats, etc.) to be cleaned and/or disinfected. The computer system 24 may further use such information to determine the optimized disinfection strategy based on previous data on cleaning and disinfecting similar objects. The biocidal efficacy and the use of Disinfectant can be optimized through, including but not limited to, controlling the amount of disinfectant use, droplet size, the spraying pressure (thus controlling the Disinfectant droplet travel distance), the disinfection time and the angle of which the Disinfectant is applied.

The computer system 24 may include a microprocessor (not shown) and a computer-readable storage medium or memory (not shown) connected to the microprocessor.

In some embodiments, the computer system 24 controls the disinfection module to continue to dispense the Disinfectant until the disinfectant sensor 18 measures the concentration of the Disinfectant surrounding air reaches a predetermined level.

In some embodiments, the opening size of the nozzle 110 is ranged from 10 nm to 80 nm. In some specific embodiments, the opening sizes of the nozzle 110 is 10 nm, 20 nm, 40 nm or 80 nm.

In some embodiments, the robot 100 further includes an extendable robotic arm to extend the electrostatics sprayer 108 away from the main body of the robot. In one specific embodiment, the arm may extend the electrostatics sprayer 108 from 0.1 to 5 meters away from the main body of the robot 100. In some embodiments, the robotic arm extends the electrostatics sprayer 108 toward the ceiling.

In some embodiments, the concentration of the Disinfectant may be adjusted based on the specific application (in some circumstances, detergent may be added for particular purpose). In some embodiments, an adherence chemical (e.g. polymer) may be added into the Disinfectant to allow it to stay on the surface longer.

In some embodiments, the robot 100 may include any rechargeable battery. In yet some embodiment, the robot includes lithium battery.

In some embodiments, the robot 100 may include motor configured to drive the wheel 14.

In some embodiments, the obstacle sensor may be a medium range scanner and may give the robot 100 a 360 degree visibility for at least 5 meters. In some embodiments, the scanner may be light sourced (e.g. laser) or an audio-based (e.g. sonar). In some embodiments, the obstacle sensor may be a camera and/or sonar transducer.

In some embodiments, the disinfection sensor 18 may be detachable from the robot 100 and communicates with the robot 100 wirelessly. In some embodiments, the disinfection sensor 18 may comprises an optical sensor (e.g. camera) and a compartment capable of holding a chemical indicator and exposing it to the surrounding environment. The chemical indicator may change its color upon the Disinfectant reaches a predetermined concentration. The optical sensor picks up the color change of the chemical indicator and send a signal to the robot 100. In yet some other embodiments, the concentration of the Disinfectant is measured using electronics means, for example, change of current or voltage upon the Disinfectant reaches a predetermined concentration.

The robot 100 may further connected to a communication network through a communication module 112 according to one embodiment. The communication network may further connect to a remote computer system where user may instruct the robot 100 from a remote location. In yet some particular embodiments, the robot 100 may further communicate with the other robot 100 through its communication module 112. For example, the robot 100 may share its cleaning and disinfection parameters, including but not limited to the data received by its sensors, with the other robot 100 and/or the communication network.

In some embodiments, the wireless communication may include a WI-FI hotspot network hosted by a router or the robot 100. In another embodiment, the wireless communication may include a combination of the WI-FI hotspot network 204 and a cellular network. In a further embodiment, the wireless communication may include satellite communication. In yet another example, the wireless communication may include BLUETOOTH, NFC, or other short-range wireless communication protocols where the robot 100 may communicate with another robot 100. In yet another example, the wireless communication may be any wireless and/or wired communication protocols.

In some embodiments, the Disinfectant is sprayed in an aerosol level dry fog form.

In some embodiments, the robot 100 further comprises a foldable telescope handle. The user can push, pull or drag the robot 100 by hand.

In some embodiments, the robot 100 further comprises an audible alarm for alerting the user of any alarming situations and an emergency stop button to terminate any of its operation when there is emergency.

In some embodiments, the robot 100 further comprise air compressor to provide pressurized gas for the spraying pressure.

In some embodiments, the robot 100 has two fluid storages for optionally holding different Disinfectants. For example, one fluid storage for holding prewash or disinfectant and the other fluid storage holding QACs. In yet some embodiments, the robot 100 has more than two fluid storages.

In some embodiments, the weight of the robot 100 is less than 40 kg without Disinfectant.

In some embodiments, the width of the robot 100 is smaller than 15 inches.

In some embodiments, the electrostatic sprayer 108 may be detachably separated from the main body of the robot and the Disinfectant is supplied to the electrostatic sprayer 108 through a flexible hose. In some embodiments, the electrostatic sprayer 108 may be detachable connected to the mechanical arm.

In some embodiments, the wheel 14 of the mobile robot may be all-wheel drive. In some other embodiments, the wheel 14 is a continuous track to help the robot 100 to operate on a non-flat ground, e.g., door threshold, bumps or ramps.

In some embodiments, the each elements of the robot 100 may be detachably separated for easy deployment. For example, the main body of the robot 100 may be detachably separated from the motor and the wheel 14.

In some embodiment, the robot 100 may perform self-cleaning protocol to disinfect itself immediately after the disinfection to protect the cleaning operator from contamination. The self-cleaning protocol may be perform my spraying Disinfectant around the environment quickly such that the Disinfectant can land on its robot 100 body to disinfect it.

### Third Embodiment

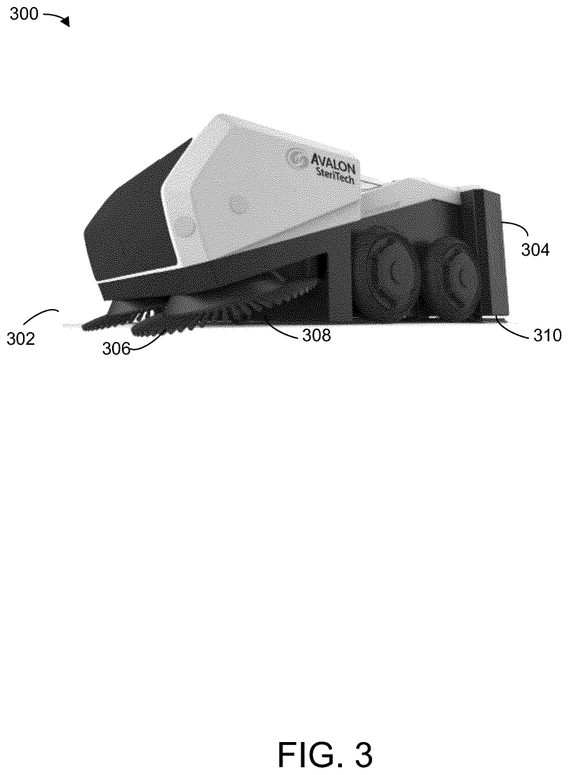

Now refer to the automatic cleaning and disinfection robot 300 according to a second embodiment of the present invention. The robot 300 in this embodiment is specific for cleaning and disinfecting HVAC conduit. As shown in FIG. 3, the robot of this specific embodiment comprises at least one fluid storage configured to hold Disinfectant, at least one obstacle sensor installed at the front of the robot configured to detect any obstacle in its path as it travels, at least one disinfectant sensor configured to obtain the amount of Disinfectant dispensed to the surrounding environment, a cleaning module 302 configured to remove visible foreign matters from a surface, a disinfection module 304 configured to spray the Disinfectant onto surface around the robot 300, and a computer system configured to receive the signals from the sensors, control the disinfection module 304 based on the signal from the disinfectant sensor, and navigate through a predetermined area while avoiding obstacle based on the signal from the obstacle sensor.

The cleaning module 302 comprises two rotatable brushes 306 are inclined such that the one side of the brush could contact the surface to be cleaned. The left rotatable brush is configured to rotate such that the brushes on those two rotatable brushes may sweep, push and/or direct an object on the surface in front of the mobile robot toward the mobile robot.

The cleaning module further comprises the vacuum system comprises a vacuum mouth 308 disposed around the rotatable brushes and a compartment configured to capture and store the trash entering the vacuum mouth. The vacuum mouth 308 is configured to capture trash in different size. In some embodiment, the trash range from dust to a typical plastic water bottle. In some embodiment, the compartment further comprises a filter bag such that all the object are captured and stored within such bag for easy removal and clean up by the cleaning operator.

The disinfection module 304 comprises a fabric mat 310 installed at the bottom of the mobile robot 300. Such fabric mat is installed onto a retractable platform and facing the surface under the robot 300. The disinfection module 304 further comprises spraying system configured to dispense the liquid in the fluid storage onto the fabric mat and/or the surface under the robot 300.

The disinfectant sensor detects the amount of Disinfectant dispensed onto the mat. If the mat does not have enough amount Disinfectant, the robot will move slower or stop moving until the amount of Disinfectant reaches a predetermined level.

### Forth Embodiment

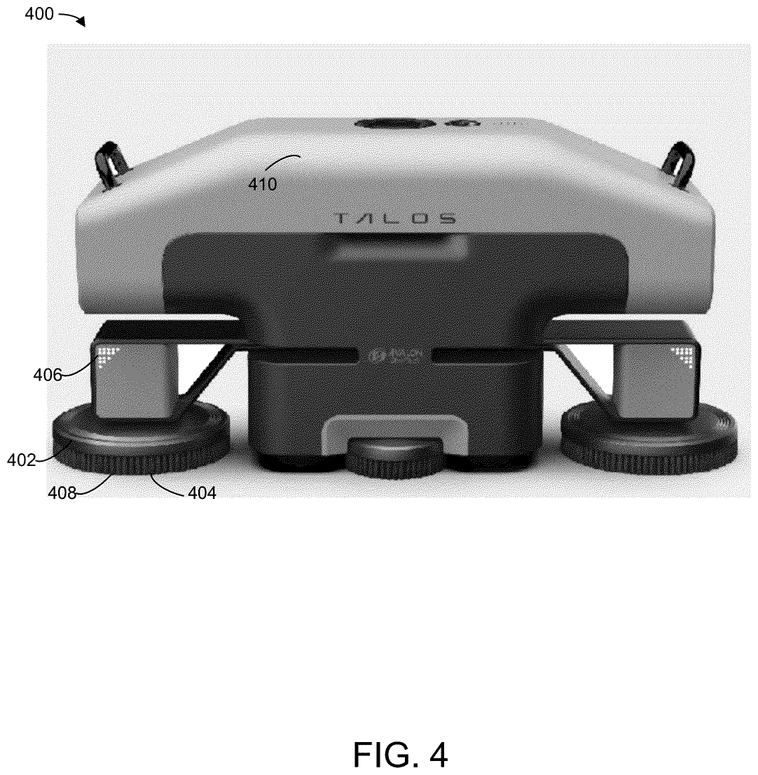

Now refer to the automatic cleaning and disinfection robot 400 according to a second embodiment of the present invention. The robot 400 in this embodiment is specific for cleaning and disinfecting floor, in particular, scrubbing floor. As shown in FIG. 4, the robot 400 of this specific embodiment comprises at least one fluid storage configured to hold Disinfectant, at least one obstacle sensor installed at the front of the robot configured to detect any obstacle in its path as it travels, at least one disinfectant sensor configured to obtain the amount of Disinfectant dispensed to the surrounding environment, a cleaning module 402 configured to remove visible foreign matters from a surface, a disinfection module 404 configured to spray the Disinfectant into the surface around the robot 400, and a computer system configured to receive the signals from the sensors, control the disinfection module based on the signal from the disinfectant sensor, and navigate through a predetermined area while avoiding obstacle based on the signal from the obstacle sensor.

The cleaning module 402 comprises at least one mechanic arm 406 and a rotatable brush 408 disposed on the mechanic arm. The mechanic arm 406 is configured to push the rotatable brush 408 toward the floor during scrubbing and retracts the rotatable brush 408 when the robot travels. The mechanic arm 406 may extend the rotatable brush 408 to the floor around the robot to scrubs floor.

The disinfection module 404 comprises spraying nozzle disposed at the center of the brush and configured to dispense the liquid in the fluid storage 410 onto the rotatable brush 408 and/or the surface under the rotatable brush 408.

The disinfectant sensor detects the amount of Disinfectant dispensed onto the area of the brush 408. If such area does not have enough Disinfectant, the robot will increase the scrubbing time.

Now turn to the operation of the robot 100. Upon deployment of the robot 100, the robot 100 may begin release of the Disinfectant into the environment to disinfect a portion or a section of the environment (e.g. public transits, including but not limited to aeroplane, railway, ferry, ship, bus, van, car, conduits, etc). The disinfectant sensor 18 may monitor and focus the detection of concentration of the Disinfectant. Upon detecting or sensing the concentration of the Disinfectant reaches a predetermined level, the release of the Disinfectant may stop. As discussed above, the robot 100 may then move to the next spot.

In such an example, once the disinfectant sensor 18 has provide a positive signal to the computer system, the computer system may then move to next position or perform further disinfection based on the object it is disinfecting due to previous disinfection data on disinfecting similar object. The deployment may be completed once the entire environment has been disinfected.

In yet another embodiment, more than one robot 100 may be deployed to the same environment at the same time to increase efficiency and reduce time to disinfect. Each robot 100 may communicate each other and share data in connection with the cleaning and disinfection through a wireless network.

In yet another embodiment, the obstacle sensor 16 may track certain image (e.g. QR code) and follow the tracked image.

It is to be understood that other means to spray the antimicrobial solution may be provided without departing from the spirit and scope of the embodiments. In another example, a separate fluid storage may be used to hold the antimicrobial solution.

The example embodiments may include additional devices and networks beyond those shown. Further, the functionality described as being performed by one device may be distributed and performed by two or more devices. Multiple devices may also be combined into a single device, which may perform the functionality of the combined devices.

The various participants and elements described herein may operate one or more computer apparatuses to facilitate the functions described herein. Any of the elements in the above-described Figures, including any servers, user devices, or databases, may use any suitable number of subsystems to facilitate the functions described herein.

Any of the software components or functions described in this application, may be implemented as software code or computer readable instructions that may be executed by at least one processor using any suitable computer language such as, for example, Java, C++, or Python using, for example, conventional or object-oriented techniques.

The software code may be stored as a series of instructions or commands on a non-transitory computer readable medium, such as a random access memory (RAM), a read only memory (ROM), a magnetic medium such as a hard-drive or a floppy disk, or an optical medium such as a CD-ROM. Any such computer readable medium may reside on or within a single computational apparatus and may be present on or within different computational apparatuses within a system or network.

It may be understood that the present invention as described above may be implemented in the form of control logic using computer software in a modular or integrated manner. Based on the disclosure and teachings provided herein, a person of ordinary skill in the art may know and appreciate other ways and/or methods to implement the present invention using hardware, software, or a combination of hardware and software.

The above description is illustrative and is not restrictive. Many variations of embodiments may become apparent to those skilled in the art upon review of the disclosure. The scope embodiments should, therefore, be determined not with reference to the above description, but instead should be determined with reference to the pending claims along with their full scope or equivalents.

One or more features from any embodiment may be combined with one or more features of any other embodiment without departing from the scope embodiments. A recitation of “a”, “an” or “the” is intended to mean “one or more” unless specifically indicated to the contrary. Recitation of “and/or” is intended to represent the most inclusive sense of the term unless specifically indicated to the contrary.

One or more of the elements of the present system may be claimed as means for accomplishing a particular function. Where such means-plus-function elements are used to describe certain elements of a claimed system it may be understood by those of ordinary skill in the art having the present specification, figures and claims before them, that the corresponding structure includes a computer, processor, or microprocessor (as the case may be) programmed to perform the particularly recited function using functionality found in a computer after special programming and/or by implementing one or more algorithms to achieve the recited functionality as recited in the claims or steps described above. As would be understood by those of ordinary skill in the art that algorithm may be expressed within this disclosure as a mathematical formula, a flow chart, a narrative, and/or in any other manner that provides sufficient structure for those of ordinary skill in the art to implement the recited process and its equivalents.

While the present disclosure may be embodied in many different forms, the drawings and discussion are presented with the understanding that the present disclosure is an exemplification of the principles of one or more inventions and is not intended to limit any one embodiments to the embodiments illustrated.

Further advantages and modifications of the above described system and method may readily occur to those skilled in the art.

The disclosure, in its broader aspects, is therefore not limited to the specific details, representative system and methods, and illustrative examples shown and described above. Various modifications and variations may be made to the above specification without departing from the scope or spirit of the present disclosure, and it is intended that the present disclosure covers all such modifications and variations provided they come within the scope of the following claims and their equivalents.

What is claimed is:

1. An automated cleaning and disinfection robot comprising:
an automated navigation system capable of navigating through an enclosed area and reaching predetermined spots thereof while avoiding obstacles;
a fluid storage for storing Quaternary Ammonium Compounds (QACs) and/or other disinfectant (“Disinfectant”), wherein an adherence additives may be added to such Disinfectant to enhance the ability for allowing Disinfectant to adhere to a surface thus providing extra and prolonged protection against the pathogen agents;
a plurality of sensors to
(i) detect obstacles;
(ii) identify contaminated spot; and
(iii) measure the content of biocidal in the surrounding area to ensure
striking the perfect balance between biocidal efficacy and over-use;
a flexible propulsion system that can move the robot through different space within a public transits;
a disinfection module capable of removing contaminates and pathogenic agents on a predetermined surface; and
a non-transitory computer readable storage medium configured to store instructions that when executed is configured to cause a processor to execute the instructions for:
(i) obtaining at least one environment parameter including at least the temperature, the humidity and the pressure, and
(ii) optimizing biocidal efficacy and the amount of Disinfectant used based on the environment parameter,
wherein the robot is configured to
(i) operate in an environmentally friendly approach; and
(ii) prevent contamination/cross infection due to the prolonged adherence of the Disinfection chemical on a surface.

2. The automated cleaning and disinfection robot of claim 1, wherein the disinfection module comprises:
an atomization system energized by pressurized air, wherein the spraying pressure helps create Disinfectant droplet;
an electrostatic sprayer having an nozzle with an opening size selected from 10, 20, 40 or 80 nm for controlling the droplet size sprayed into the air as well as onto a surface;
a pressure control unit to control the spraying pressure thereby controlling the distance and/or height traveled by the droplets; and
an optional mechanical arm configured to extend the electrostatic sprayer away from the robot,
wherein the combination of the nozzle and the pressure control unit helps reducing the amount of Disinfectant used, and
wherein the concentration of the Disinfectant may be adjusted based on the specific application.

3. The automated cleaning and disinfection robot of claim 2, wherein optimizing the biocidal efficacy and the amount of Disinfectant used involve the step of controlling at least one of the following: the droplet size, the spraying pressure (thus controlling the Disinfectant droplet travel distance), the disinfection time and the angle of which the Disinfectant is applied.

4. The automated cleaning and disinfection robot of claim 3, wherein optimizing the biocidal efficacy and the amount of Disinfectant used further involve the step of classifying the surrounding materials and/or object to be disinfected and optimize the biocidal efficacy based on the materials and/or objects by controlling at least one of the following: the spraying pressure (thus controlling the Disinfectant droplet travel distance), the disinfection time and the angle of which the Disinfectant is applied.

5. The automated cleaning and disinfection robot of claim 2 wherein the mechanical arm extends the electrostatic sprayer ranged from 0.1 to 5 meters away from the robot.

6. The automated cleaning and disinfection robot of claim 2, wherein the robot is configured for performing cleaning and disinfection in railway system.

7. The automated cleaning and disinfection robot of claim 2, wherein the robot is configured to spray the droplet ranged from 0.1 to 30 meters from the electrostatic sprayer.

8. The automated cleaning and disinfection robot of claim 2, wherein the robot is configured to clean and disinfect an enclosed space in a more environmental friendly approach and configured to help preventing cross infection.

9. The automated cleaning and disinfection robot of claim 1, further comprising
a cleaning module comprising
a vacuum having a vacuum opening to vacuum large trash objects; and
a plurality of brushes to sweep large trash objects towards the vacuum opening,
wherein the disinfection module comprises a mop to map a surface on the HVAC conduit with Disinfectant.

10. The automated cleaning and disinfection robot of claim 1, further comprising
a cleaning module comprising
at least one brush for scrubbing a surface; and
a mechanical arm for moving the brush in various directions to increase the scrubbing area,
wherein the disinfection module comprises a dispenser for dispensing the Disinfectant onto a surface where the brush scrubs.
